(19) United States
(12) Patent Application Publication
(10) Pub. No.: US 2006/0106227 A1
(43) Pub. Date: May 18, 2006

(54) 3-'2-(Dimethylamino) ethyl!-n-methyl-1h-indole-5-methanesulfonamide and the succinate thereof

(75) Inventors: Manne Satyanarayana Reddy, Hyderabad (IN); Srinivasan Thirumalai Rajan, Hyderabad, IN (US); Mokkarala Suryanarayana Murthy, Hyderabad, IN (US); Achampeta Kodanda Ram Prasad, Hyderabad, IN (US)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignee: Dr Reddy's laboratories Limited, Hyderabad 500 016, Andhra Pradesh (IN)

(21) Appl. No.: 10/516,897

(22) PCT Filed: Jun. 16, 2004
(86) PCT No.: PCT/US04/19004

§ 371 Date: Jul. 5, 2005

(30) Foreign Application Priority Data
Jun. 13, 2002 (IN) 451/MAS/02
Jun. 13, 2002 (IN) 452/MAS/02

(51) Int. Cl. C07D 209/10

(52) U.S. Cl. 548/507

(57) ABSTRACT

A process for the preparation of highly pure Sumatriptan id described. A process for the preparation of novel crystalline Form I and crystalline Form II of Sumatriptan succinate is described. Sumatriptan is used for alleviating the pain of migraine headaches.

### FIELD OF INVENTION

Sumatriptan succinate, is a known anti-migraine agent and marketed under the brand name “Imitrix” in US market.

The present invention relates to the novel crystalline forms of 3-[2-(Dimethylamino)ethyl]-N-methyl-IH-indole-5-methane sulfonamide succinate (Sumatriptan succinate). The present invention also relates to the process for the preparation of novel crystalline forms of 3-[2-(Dimethylamino)ethyl]-N-methyl-IH-indole-5-methane sulfonamide succinate (Sumatriptan succinate), which can be depicted as FIG. (1). 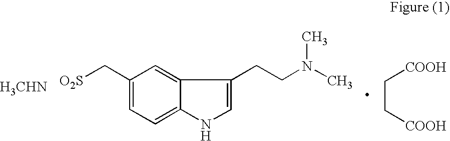
Sumatriptan and its pharmaceutically related salts are therapeutically used as anti migraine agents and for the treatment of cluster headaches.

### SUMMARY OF THE INVENTION

A process for the preparation of Sumatriptan Succinate comprises:

1) reacting a solution of 3-(2-aminoethyl)-N-methyl-1H-indole-5-methane sulphonamide in a lower alkanol such as methanol with solution of a reducing agent such as sodiumborohydride in water and formalin in a lower alkanol such as methanol. Then an acid such as hydrochloric acid is added to the resulting reaction mixture, followed by treatment with a basifying agent such as potassium carbonate and extracting with a polar or organic solvent such as ethyl acetate to get the Sumatriptan base compound.

2) reacting the solution of N-methyl-1H-indole-5-methanesulphonamide in an anhydrous polar solvent such as tetrahydrofuran with oxalyl chloride under nitrogen atmosphere, gaseous dimethylamine in N,N-Dimethyl-3-[2-(mehylamino)sulphonyl]methyl]-oxo-1H-indole-3-acetamide, which is refluxed with a reducing agent such as lithium aluminum hydride in tetrahydrofuran under an inert atmosphere such as nitrogen, followed by the addition of a base such as sodium hydroxide to result in the Sumatriptan base and it is converted to its corresponding succinate salt.

The pharmaceutical industry has intensified its studies on polymorphism in drugs, and the difference in the activity of different polymorphic forms of a given drug. This has especially become very interesting after observing that many antibiotics, antibacterial, tranquillizers etc. exhibit polymorphism and one or some of the polymorphic forms of a given drug exhibit superior bioavailability and consequently show much higher activity compared to other polymorphs.

Since Sumatriptan succinate is useful as an anti migraine drug, there is a need to produce Sumatriptan succinate in a pure and crystalline form to enable formulations to meet exacting pharmaceutical requirements and specifications.

Furthermore, the process by which it is produced needs to be one which is amenable to large-scale production.

Further it is desirable that the formulation processes be facilitated by use of the active crystalline materials that are free flowing high melting solids. Such free flowing high melting solids are easier to handle than amorphous solids.

Finally, it is economically desirable that the product should be stable for extended periods of time without need for specialized storage conditions.

We have now surprisingly and unexpectedly found that Sumatriptan succinate can be produced in two crystalline forms. The crystalline forms of present invention are high melting solids, very suited for formulation.

The crystalline forms of Sumatriptan succinate of present invention are designated as Form-I and Form-II for convenience and hereinafter, these are referred as Crystalline Form-I and Form-II of Sumatriptan succinate.

Crystalline Form-I and Form-II of present invention have been prepared from highly pure Sumatriptan base and have resulted in pure crystalline forms of sumatriptan succinate.
Sumatriptan was repeatedly recrystallized in acetone to obtain the highly pure sumatriptan. The basic procedure is as follows:
A process for the preparation of highly pure N-Methyl-3-[2-(dimethylamino) ethyl]-1H-Indole-5-methane sulfonamide (Sumatriptan), which comprises;
   a. dissolving crude Sumatriptan in acetone at reflux temperature to a clear solution;
   b. treating the obtained clear solution with charcoal;
   c. concentrating the clear filtered solution to about filterable volume level;
   d. cooling the reaction mixture to a temperature of 0-30° C.; and
   e. filtering the obtained solid by conventional methods.

The process of repeated crystallization is as follows:
a. dissolving crude Sumatriptan in Acetone at reflux temperature to a clear solution;
b. treating the obtained clear solution with charcoal;
c. concentrating the clear filtered solution to about filterable volume level;
d. cooling the reaction mixture to a temperature of about 0-30° C., preferably 0-5° C.;
e. filtering the obtained solid by conventional methods;
f. dissolving the obtained wet material from step (e) in Acetone at reflux temperature to a clear solution;
g. treating the obtained clear solution with charcoal;
h. concentrating the clear filtered solution to about filterable volume level;
i. cooling the reaction mixture to a temperature of about 0-30° C., preferably 0-5° C.;
j. filtering the obtained solid by conventional methods;
k. dissolving the obtained wet material from step (j) in Acetone at reflux temperature to a clear solution;
l. filtering the clear solution obtained;
m. concentrating the clear filtered solution to about filterable volume level;
n. cooling the reaction mixture to a temperature of 0-30° C., preferably 0-5° C.;
o. filtering the obtained solid by conventional methods and accompanied by drying the compound at a temperature of 30-100° C., preferably 50-60° C. to afford the desired highly pure Sumatriptan.

The highly pure Sumatriptan obtained in the above process has the following characteristics:
HPLC Purity levels of above 99%, more preferably above 99.5%, more preferably above 99.7%.
Any unknown impurity at a level of NMT 0.1%

The present inventive substance of highly pure Sumatriptan was analyzed by High performance liquid chromatography (HPLC) to know the purity levels. The HPLC method followed is in the European pharmacopoeia 4^{th} Edition 2002, pages 1991-1993, the subject matter of which is incorporated herein by reference.

The highly pure Sumatriptan obtained as per the procedure described above was further characterized by X-ray diffractogram and Infrared spectrum.

The characteristic X-ray diffractogram and Infrared spectrum of a sample of highly pure Sumatriptan is substantially as depicted in FIG. (7) and FIG. (8) respectively.

The obtained highly pure Sumatriptan of the present invention may be converted into its pharmaceutically acceptable salts, preferably Succinate salt as described below. Thus, obtained Sumatriptan succinate is highly pure and well suited for pharmaceutical formulations.

Another beneficial aspect of the present invention is that, the Sumatriptan succinate is obtained in almost quantitative yield from the precursor i.e., Sumatriptan.

The highly pure Sumatriptan of the present invention can be converted to succinate salt in situ and used for injectable pharmaceuticals.

Hence, the present invention is a simple, cost-effective and environmentally friendly process.

The present invention is illustrated by the following examples, which are not intended to limit the effective scope of the claims.

The crystalline Form I and Form II of Sumatriptan succinate have been characterized by X-ray powder diffractogram, Differential Scanning Colorimetry thermogram and Infra red spectra.

The processes of preparing crystalline Forms I and II are simple, eco-friendly and easily scaled up to large sized reactions.

The process for the preparation of crystalline Form-I of Sumatriptan succinate comprises, treating Sumatriptan in polar solvents such as ketones or ethers or esters or alcohols followed by addition of Succinic acid at reflux temperature and further cooling to ambient temperature to get the desired crystalline form.

The process for the preparation of crystalline Form-II of Sumatriptan succinate comprises, treating the Sumatriptan in aliphatic/alicyclic hydrocarbon solvents or halo solvents such as cyclohexane or dichloromethane followed by addition of Succinic acid at reflux temperature and further cooling to ambient temperature to afford the desired novel crystalline form.

### BRIEF DESCRIPTION OF THE DRAWINGS

FIG. 1 is an XRD pattern of a sample of crystalline Form-I of present invention.

FIG. 2 is a DSC thermogram of a sample of crystalline Form-I of the present invention.

FIG. 3 is an IR spectrum of a sample of crystalline Form-I of the present invention.

FIG. 4 is an XRD pattern of a sample of crystalline Form-II of present invention.

FIG. 5 is a DSC thermogram of a sample of crystalline Form-II of the present invention.

FIG. 6 is an IR spectrum of a sample of crystalline Form-II of the present invention.

FIG. 7 is an example of XRD pattern of a sample of a highly pure Sumatriptan.

FIG. 8 is an IR spectrum of sample of highly pure Sumatriptan.

### DETAILED DESCRIPTION OF INVENTION

As used herein, the term “highly pure” means at least about 99% pure by HPLC, more preferably at least about 99.5% pure by HPLC, most preferably at least about 99.7% pure by HPLC. The HPLC method followed was as mentioned in the European Pharmacopoeia, 4^{th} Edition 2002, pages 1991-1993, the subject matter of which is incorporated herein by reference.

The present invention also relates to the crystalline Form-I and Form-II of Sumatriptan succinate and a process for the preparation thereof.

Crystalline Form-I and Form-II of Sumatriptan succinate of the present invention are characterized by their X-ray diffractogram, Differential Scanning colorimetry thermogram and IR spectrum.

The X-ray diffraction patterns of Form-I and Form-II of Sumatriptan succinate were measured on a Bruker Axe, DS Advance Powder X-ray Diffractometer with Cu K alpha-1 Radiation source.

The 2-theta values and the intensity percentages of relevant peaks in X-ray powder diffraction patterns of a sample crystalline Form-I and a sample Form-II of Sumatriptan succinate is shown in the Table-1.

TABLE 1

| | |
|---|---|
| Form-I | Form-II |

| 2-Theta | Intensity | 2-Theta | Intensity |
|---|---|---|---|
| Values (°) | I/Iₒ (%) | Values (°) | I/Iₒ (%) |
| 15.704 | 100.00 | 19.966 | 100.0 |
| 16.397 | 97.1 | 26.089 | 66.1 |
| 20.582 | 54.0 | 7.320 | 47.2 |
| 16.198 | 45.3 | 22.904 | 26.6 |
| 20.061 | 36.1 | 17.495 | 23.2 |
| 15.412 | 29.7 | 20.615 | 23.0 |
| 21.353 | 29.5 | 31.474 | 19.3 |
| 22.734 | 24.5 | 16.406 | 18.3 |
| 19.894 | 20.2 | 14.707 | 18.1 |
| 13.256 | 19.4 | 22.082 | 17.7 |
| 26.938 | 18.0 | 16.202 | 16.4 |
| 20.243 | 17.1 | 19.047 | 15.3 |
| 12.628 | 17.0 | 18.751 | 14.3 |
| 18.107 | 15.9 | 29.675 | 13.5 |
| 26.018 | 12.7 | 21.360 | 13.4 |
| — | — | 15.710 | 13.3 |
| — | — | 17.111 | 11.4 |
| — | — | 15.424 | 10.9 |
| — | — | 21.176 | 10.8 |

The X-ray powder diffraction pattern of the present invention of crystalline Form-I and Form-II of Sumatriptan succinate are substantially depicted in FIG. (1) and FIG. (4) respectively.

Differential Scanning Colorimetry thermograms of crystalline Form-I and Form-II of Sumatriptan succinate, were also prepared.

The crystalline Form-I and Form-II of Sumatriptan succinate were analyzed on Schimadzu differential scanning colorimeter in a temperature range of about 25 to about 230° C. with a heating rate of about 5° C./minute under nitrogen with a flow rate of about 50.0 ml/minute.

The Differential Scanning Colorimetry thermogram of crystalline Form-I of sumatriptan succinate exhibits a significant endo peak around 169° C. and substantially as depicted in FIG. (2).

The Differential Scanning Colorimetry thermogram of crystalline Form-II of sumatriptan succinate exhibits significant major endo peak around 168° C., minor endo peaks around 122° C., 160° C. and substantially as depicted in FIG. (5).

Infra red spectral data of crystalline Form-I and Form-II of Sumatriptan succinate, were measured on Perkin-Elmer FT-IR instrument by KBr-transmission method. The identified significant Infrared bands of these forms are set forth in the following Table-2.

TABLE 2

| Form-I | Form-II |
|---|---|
| Wave length | Wave length |
| (Cm⁻¹) | (Cm⁻¹) |
| 3373.63 | 3358.48 |
| 3101.60 | 3268.77 |
| 2932.85 | 2931.89 |
| 1708.32 | 1707.42 |
| 1566.39 | 1569.95 |
| 1338.95 | 1336.02 |
| 1299.80 | 1301.84 |
| 1270.21 | 1264.14 |
| 1170.81 | 1143.56 |
| 1081.92 | 1092.11 |
| 884.58 | 884.82 |
| 638.67 | 639.13 |

The relevant Infra red spectrum of the present invention of crystalline Form-I and Form-II of Sumatriptan succinate are substantially as depicted in FIG. (3) and FIG. (6) respectively.

Another embodiment of the present invention provides a process for preparing a novel crystalline Form-I of Sumatriptan succinate, which comprises;
a) treating highly pure Sumatriptan base in ketone solvents such as acetone, methyl isobutyl ketone or methyl ethyl ketone, preferably acetoneor ether solvents such as tetrahydrofuran, diethyl ether, diisopropyl ether or diisobutyl ether, preferably tetrahydrofuran; or ester solvents such as methyl acetate, ethyl acetate, propyl acetate or butyl acetate, preferably ethyl acetate; or alcoholic solvents such as methanol, propanol, isopropanol, butanol, isobutanol or mixtures thereof, preferably a mixture of methanol and isopropanol at reflux temperature;
b) adding Succinic acid to the reaction mixture;
c) optionally concentrating the reaction mixture;
d) cooling the reaction mixture to a temperature of 0-35° C., preferably 25-35° C.;
e) filtering the isolated solid by conventional techniques accompanied by drying the solid at a temperature of 50-100° C., preferably 80-90° C. to afford the crystalline Form-I of Sumatriptan succinate.

Another embodiment of the present invention provides a process for preparing novel crystalline Form-II of Sumatriptan succinate, which comprises;
a) refluxing highly pure Sumatriptan in aliphatic/alicyclic hydrocarbon solvents such as petroleum ether, n-hexane, n-heptane, cyclohexane or cycloheptane, preferably cyclohexane, or halogenated solvents such as chloroform, dichloromethane, dichloroethane or carbon tetrachloride, preferably dichloromethane;
b) adding Succinic acid to the reaction mixture;
c) stirring the reaction mixture at reflux for about 30 minutes to about 4 hours, preferably about 60 minutes;
d) cooling the reaction mixture to a temperature of about 0° to about 35° C., preferably about 25° to about 35° C.;
e) filtering the isolated solid by conventional techniques and drying the obtained solid at a temperature of about 30° to about 100° C., preferably about 70° to about 90° C. to afford the novel crystalline Form-II of Sumatriptan succinate.

The processes of the present invention are simple, and easily scaled up for commercial production and Sumatriptan succinate is obtained in pure and crystalline form to enable formulations to meet exacting pharmaceutical requirements and specifications.

Most pharmaceutical formulation processes are facilitated by use of the active materials that are free flowing high melting solids. The crystalline forms of present invention are high melting solids, very suited for formulation.

Moreover the novel crystalline polymorphs of the present invention are stable for extended periods of time without the need for specialized storage conditions such as low humidity and low temperature.

The novel crystalline forms of Sumatriptan succinate are useful as anti-migraine agents and as agents for treating cluster headaches. The pure Sumatriptan base is also useful an anti-migraine agent and as an agent for treating cluster headaches.
Sumatriptan may be prepared by the procedures disclosed for the preparation of Sumatriptan disclosed in U.S. Pat. No. 4,816,470 which is incorporated by reference. Other procedures known in the art can be used to prepare Sumatriptan. Sumatriptan was repeatedly recrystallized in acetone to obtain the highly pure sumatriptan. The basic procedure is as follows:
A process for the preparation of highly pure N-Methyl-3-[2-(dimethylamino) ethyl]-1H-Indole-5-methane sulfonamide (Sumatriptan), which comprises;
   a. dissolving crude Sumatriptan in acetone at reflux temperature to a clear solution;
   b. treating the obtained clear solution with charcoal;
   c. concentrating the clear filtered solution to about filterable volume level;
   d. cooling the reaction mixture to a temperature of 0-30° C.; and
   e. filtering the obtained solid by conventional methods.

The process of repeated crystallization is as follows:
a. dissolving crude Sumatriptan in Acetone at reflux temperature to a clear solution;
b. treating the obtained clear solution with charcoal;
c. concentrating the clear filtered solution to about filterable volume level;
d. cooling the reaction mixture to a temperature of about 0-30° C., preferably 0-5° C.;
e. filtering the obtained solid by conventional methods;
f. dissolving the obtained wet material from step (e) in Acetone at reflux temperature to a clear solution;
g. treating the obtained clear solution with charcoal;
h. concentrating the clear filtered solution to about filterable volume level;
i. cooling the reaction mixture to a temperature of about 0-30° C., preferably 0-5° C.;
j filtering the obtained solid by conventional methods;
k. dissolving the obtained wet material from step (j) in Acetone at reflux temperature to a clear solution;
l. filtering the clear solution obtained;
m. concentrating the clear filtered solution to about filterable volume level;
n. cooling the reaction mixture to a temperature of 0-30° C., preferably 0-5° C.;
o. filtering the obtained solid by conventional methods and accompanied by drying the compound at a temperature of 30-100° C., preferably 50-60° C. to afford the desired highly pure Sumatriptan.

The highly pure Sumatriptan obtained as per the procedure described above was further characterized by X-ray diffractogram and Infra red spectrum.

The characteristic X-ray diffractogram and Infra red spectrum of highly pure Sumatriptan is substantially as depicted in FIG. (7) and FIG. (8) respectively.

The present invention also envisages pharmaceutical compositions made using crystalline Form I of Sumatriptan succinate, crystalline Form II of Sumatriptan succinate and/or pure Sumatriptan base. The pharmaceutical compositions preferably include one or more of a physiologically or a pharmaceutically acceptable carrier, diluent, excipient, additive, filler, lubricant, binder, stabilizer, solvent or solvate.

The pharmaceutical composition may be in a form normally employed, such as tablets, capsules, lozenges, powders, syrups, solutions, suspensions, ointments, dragees and the like, may contain flavourants, sweetners, etc. in suitable solid or liquid carriers or diluents, or in suitable sterile media to form injectable solutions or suspensions. Such compositions typically contain from 1 to 25%, preferably 1 to 15% by weight of active ingredient, the remainder of the composition being one or more of a pharmaceutically acceptable carrier, diluent, excipient, additive, filler, lubricant, binder, stabilizer, solvent or solvate. The compositions of this invention may be made by methods and processes known to those of skill in the art.

The crystalline Forms I and II of Sumatriptan and Sumatriptan base can be administered to mammals, including man, via either oral, nasal, pulmonary, transdermal or parenteral, rectal, depot, subcutaneous, intravenous, intraurethral, intramuscular, intranasal, ophthalmic solution or an ointment. Administration by the oral route is preferred, being more convenient and avoiding the possible pain and irritation of injection. However, in circumstances where the patient cannot swallow the medication, or absorption following oral administration is impaired, as by disease or other abnormality, it is essential that the drug be administered parenterally. By either route, the dosage is in the range or about 0.01 to about 100 mg/kg body weight of the subject per day or preferably about 0.01 to about 100 mg/kg body weight of the subject per day or preferably about 0.01 to about 30 mg/kg body weight per day administered singly or as a divided dose. However, the optimum dosage for the individual subject being treated will be determined by the person responsible for treatment, generally smaller doses being administered initially and thereafter increments made to determine the most suitable dosage.

An effective amount means that amount of the drug or pharmaceutical agent that will elicit the biological or medical response of a tissue, system, human or animal sought.

The present invention will be explained in more detail by the following non-limiting examples.

The present invention is illustrated by the following examples, which are not intended to limit the effective scope of the claims.

### EXAMPLES

Reference Example for the Preparation of Sumatriptan Crude

To a mixture of 3-(2-Chloroethyl)-N-methyl-1H-indole-5-methane sulphonamide (100.0 grams), Potassium iodide (72.5 grams) and tetra butyl ammonium bromide (1.0 gram) was added an aqueous solution of dimethylamine (300.0 ml) and the reaction mixture is stirred at a temperature of 40-45° for 3-5 hours. The reaction completion was monitored by TLC method and upon completion of the reaction the reaction mixture was cooled to ambient temperature. Aqueous sodium carbonate solution (100.0 ml) was added and extracted into ethyl acetate (1×1 lit.+2×0.5 lit.). The combined ethyl acetate extractions were washed with brine solution (2×0.5 lit) and the layers were separated. The ethyl acetate layer was concentrated under vacuum to about 10-15 % of its initial volume. The concentrated reaction mass was stirred for 4-6 hours to crystallize the solid mass. The obtained solid was filtered, washed with ethyl acetate (50 ml) and dried to a constant weight to afford crude Sumatriptan.

(Weight: 40.0 grams, Purity by HPLC: 90.0%)

### Example 1

### Preparation of Highly Pure Sumatriptan

Crude Sumatriptan (30.3 kgs; prepared as per reference example) was suspended in acetone (900 lit) and heated to reflux to a clear solution. Then charcoal was added (7.5 kgs) and further stirred for about 15-30 minutes. The reaction mixture was filtered and washed with acetone (30 lit). The obtained filtered solution was concentrated under vacuum to about 60-70% of its initial volume. Then the reaction mixture was cooled to a temperature of 0-10° C. and stirred for 30-45 minutes. The separated solid was filtered and washed with chilled Acetone (30 lit). The obtained wet material was further suspended in Acetone (590 lit) and heated to reflux to a clear solution. Then charcoal was added (5.0 kgs) and further stirred for about 15-30 minutes. The reaction mixture was filtered and washed with acetone (20 lit). The obtained filtered solution was concentrated under vacuum to about 50-55% of its initial volume. Then the reaction mixture was cooled to a temperature of 0-10° C. and stirred for 30-45 minutes. The separated solid was filtered and washed with chilled Acetone (20 lit). The obtained wet material was further suspended in Acetone (590 lit) and heated to reflux to clear solution. The reaction solution was filtered and washed with acetone (50 lit). The obtained filtered solution was concentrated under vacuum to about 40-50% of its initial volume. Then the reaction mixture was cooled to a temperature of 0-10° C. and stirred for 30-45 minutes. The separated solid was filtered and washed with chilled Acetone (10 lit) and dried at a temperature of 50-60° C. to afford the desired highly pure Sumatriptan.
(Weight: 13.0 kgs, HPLC purity: 99.7%, Any unknown impurity <0.1%)

### Example 2

Pure Sumatriptan (10 grams) was suspended in acetone (150 ml) and heated to reflux temperature. Then Succinic acid was added (4.0 g) to the reaction mixture. The reaction mass was stirred at reflux temperature for 30-60 minutes. The mass was further cooled to a temperature of 25-35° C. and stirred for about 30-60 minutes. The solid mass was filtered, and dried at a temperature of 60-70° C. to afford the crystalline Form-I of Sumatriptan succinate.
(Weight: 13.1 grams)

### Example 3

Pure Sumatriptan (10 grams) was suspended in tetrahydrofuran (100 ml) and heated to reflux temperature. Then Succinic acid was added (4.0 g) to the reaction mixture. The reaction mass was stirred at reflux temperature for 30-60 minutes. The mass was further cooled to a temperature of 25-35° C. and stirred for about 30-60 minutes. The solid mass was filtered and dried at a temperature of 70-80° C. to afford the crystalline Form-I of Sumatriptan succinate.
(Weight: 12.4 grams)

### Example 4

Pure Sumatriptan (10 grams) was suspended in ethyl acetate (100 ml) and heated to reflux temperature. Then, Succinic acid (4.0 g) was added to the reaction mixture. The reaction mass was stirred at reflux temperature for 30-60 minutes. The mass was further cooled to a temperature of 25-35° C. and stirred for about 30-60 minutes. The solid mass was filtered and dried at a temperature of 60-70° C. to afford the crystalline Form-I of Sumatriptan succinate
(Weight: 13.2 grams)

### Example 5

Pure Sumatriptan (10 grams) was suspended in methanol (150 ml.) and heated to reflux temperature. Then Succinic acid (3.8 grams) was added to the reaction mixture. The reaction solution was stirred at reflux temperature for 15-30 minutes. The reaction solution was filtered off to get the particle free solution. The obtained solution was concentrated to about 60-70 % of its initial volume. Isopropanol (150 ml) was added to the reaction mass and further distilled off the solvent to 30-40% of its volume. The reaction mixture was cooled to a temperature of 25-35° C. and stirred for about 30-45 minutes. The solid mass was filtered, washed with isopropanol (20 ml) and dried at a temperature of 70-90° C. to afford the crystalline Form-I of Sumatriptan succinate.
(Weight: 12.10 grams)

### Preparation of Crystalline Form-II of Sumatriptan succinate

### Example 6

Pure Sumatriptan base (10 grams) was suspended in cyclohexane (150 ml) and heated to reflux temperature. Then Succinic acid (4.0 g) was added to the reaction mixture. The reaction mixture was stirred at reflux temperature for 30-60 minutes. The solution was further cooled to a temperature of 25-35° C. and stirred for about 30-60 minutes. The solid mass was filtered and dried at a temperature of 70-80° C. to afford the crystalline Form-II of Sumatriptan succinate.
(Weight: 12.0 grams)

### Example 7

Pure Sumatriptan base (10 grams) was suspended in dichloromethane (100 ml) and heated to reflux temperature. Then Succinic acid was added (4.0 g) to the reaction mixture. The reaction mixture was stirred at reflux temperature for 30-60 minutes. The solution was cooled to a temperature of 25-35° C. and stirred for about 30-60 minutes. The solid mass was further filtered, washed with dichloromethane (10 ml) and dried at a temperature of 70-80° C. to afford the crystalline Form-II of Sumatriptan succinate.
(Weight: 12.0 grams)

### DETAILED DESCRIPTION OF THE ACCOMPANYING DRAWINGS

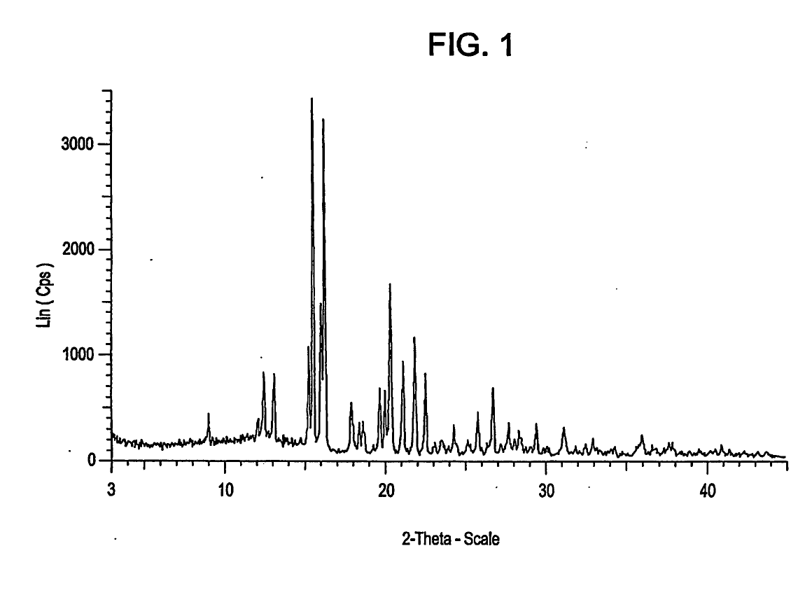

FIG. 1 is a characteristic X-ray powder diffraction pattern of a sample of crystalline Form-I of Sumatriptan succinate.

Vertical axis: Intensity (CPS); Horizontal axis: Two Theta (degrees).

The significant 2-theta values are 12.628, 13.256, 15.412, 15.704, 16.198, 16.397, 18.107, 19.894, 20.061, 20.243, 20.582, 21.353, 22.734, 26.018 and 26.938 degrees.

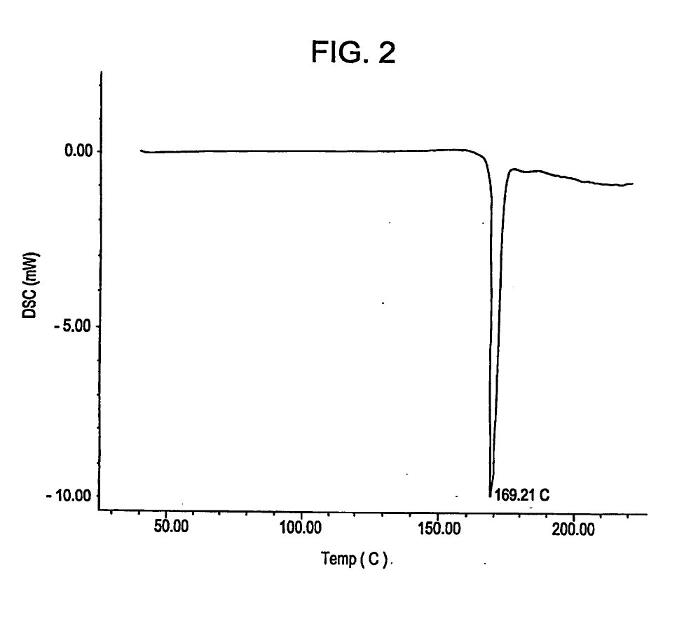

FIG. 2 is a characteristic of differential scanning calorimetry thermogram of a sample of crystalline Form-I of Sumatriptan succinate.

Vertical axis: mW; Horizontal axis: Temperature (° C.).

The differential scanning calorimetry thermogram exhibits a significant endo peak at 169.3° C.

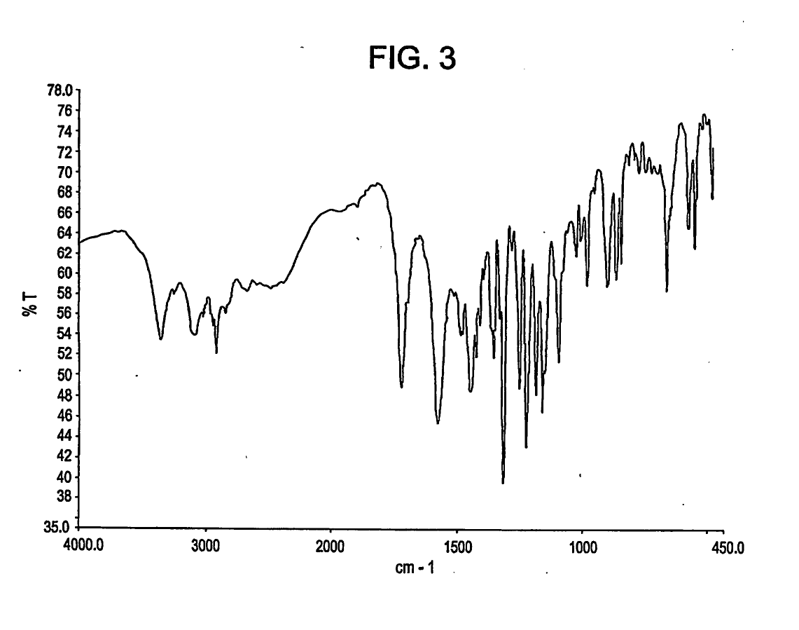

FIG. 3 is a characteristic infrared absorption spectrum of a sample of crystalline Form-I of Sumatriptan succinate.

[Vertical axis, Tramission (%); Horizontal axis: Wave number (cm⁻¹)].

The characteristic identified IR bands are around 3373, 3101, 2932, 1708, 1566, 1338, 1299, 1270, 1170, 1081, 884 and 638 cm⁻¹.

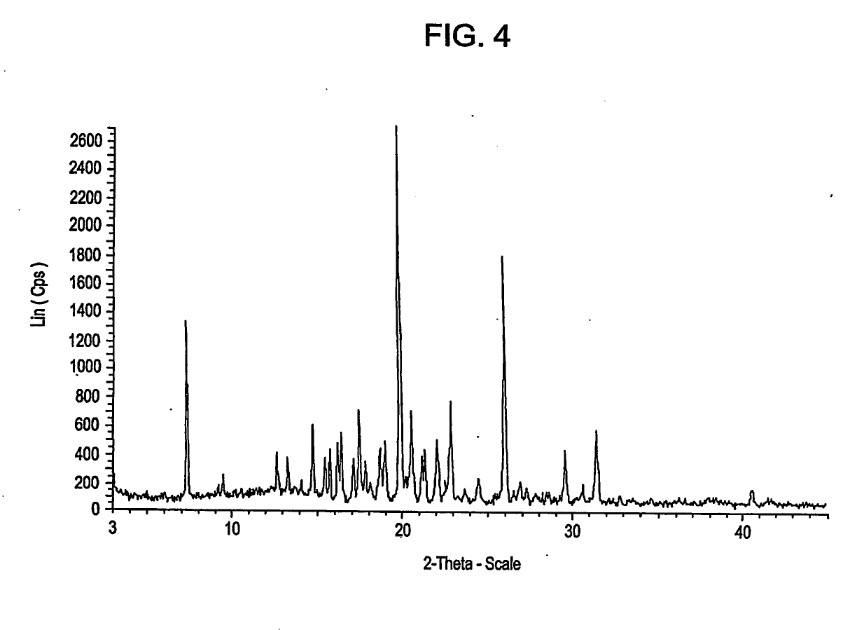

FIG. 4 is a characteristic X-ray powder diffraction pattern of a sample of crystalline Form-II of Sumatriptan succinate. Vertical axis: Intensity (CPS); Horizontal axis: Two Theta (degrees). The significant 2-theta values are around 7.320, 14.707, 15.424, 15.710, 16.202, 16.406, 17.111, 17.495, 18.751, 19.047, 19.966, 20.615, 21.176, 21.360, 22.082, 22.904, 26.089, 29.675 and 31.474 degrees.

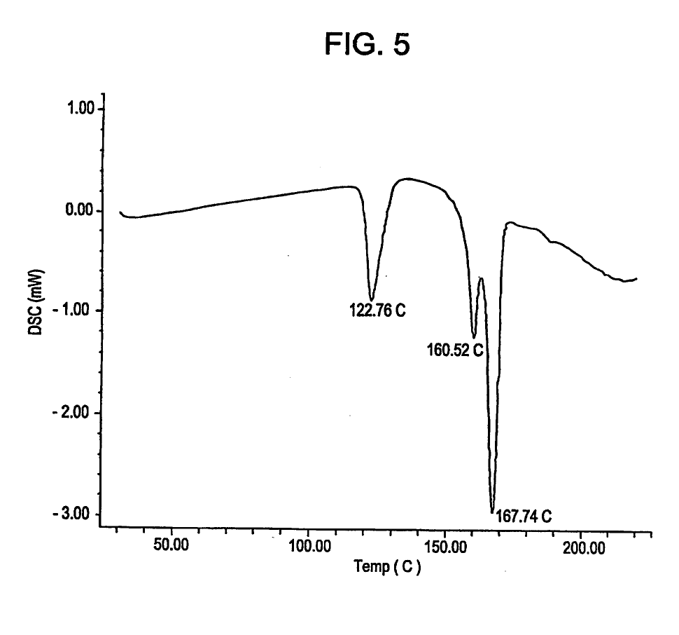

FIG. 5 is a characteristic of differential scanning calorimetry thermogram of a sample of crystalline Form-II of Sumatriptan succinate.

Vertical axis: mW; Horizontal axis: Temperature (° C.).

The differential scanning calorimetry thermogram exhibits significant major endo peak at 167.74C, minor endo peaks at 122° C. and 160° C.

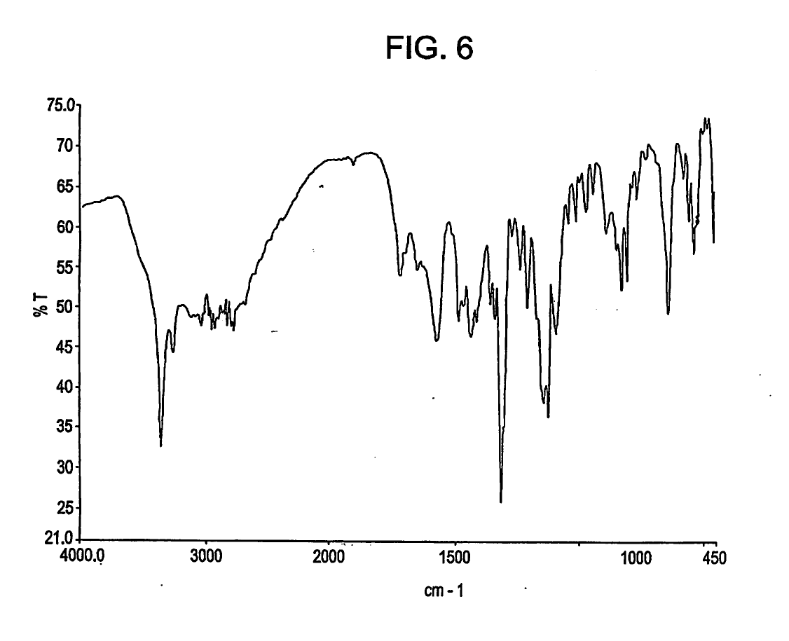

FIG. 6 is a characteristic infrared absorption spectrum of crystalline Form-II of a sample of Sumatriptan succinate.

Vertical axis, Tramission (%); Horizontal axis: Wave number (cm⁻¹)].

The characteristic identified IR bands are around 3358, 3268, 2931, 1707, 1569, 1336, 1301, 1264, 1143, 1092, 884 and 639 cm⁻¹.

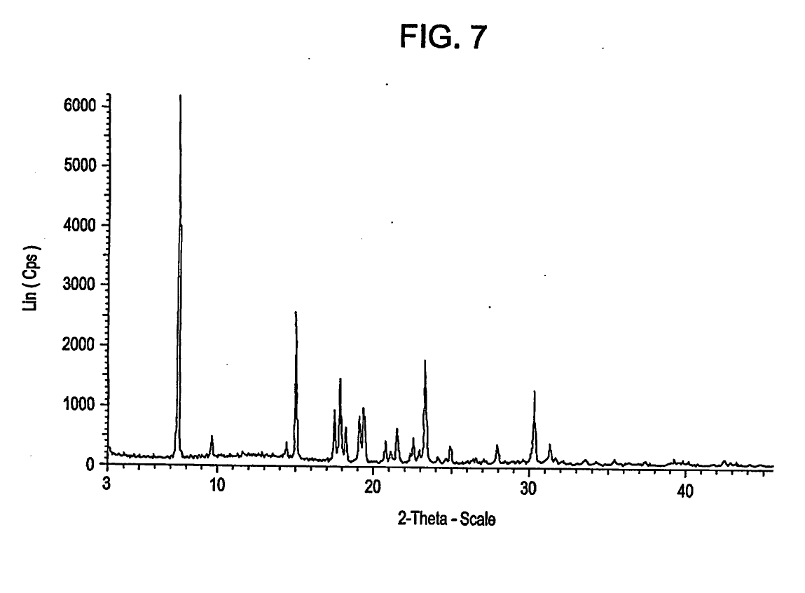

FIG. 7 is a characteristic X-ray powder diffraction pattern of a sample of highly pure Sumatriptan.

Vertical axis: Intensity (CPS); Horizontal axis: Two Theta (degrees).

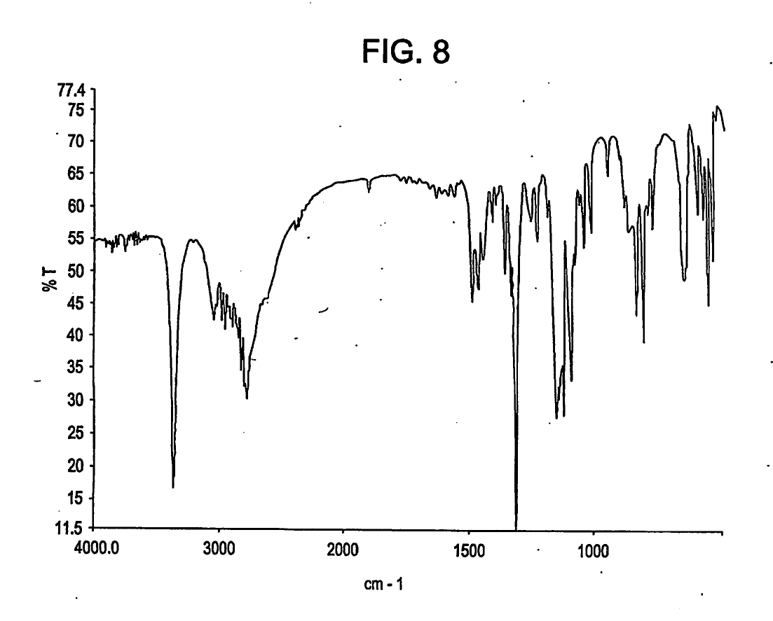

FIG. 8 is a characteristic infrared absorption spectrum of a sample of highly pure Sumatriptan.

Vertical axis, Transmission (%); Horizontal axis: Wave number (cm⁻¹)].

1. A crystalline Form-I of Sumatriptan succinate.

2. A crystalline Form-I of Sumatriptan Succinate according to claim 1 having X-ray powder diffraction pattern with peaks about 12.628, 13.256, 15.412, 15.704, 16.198, 16.397, 18.107, 19.894, 20.061, 20.243, 20.582, 21.353, 22.734, 26.018 and 26.938 two-theta degrees.

3. A crystalline Form-I of Sumatriptan succinate of claim 1 which has X-ray powder diffraction pattern substantially as depicted FIG. (1).

4. A crystalline Form-I of Sumatriptan succinate of claim 1 which has a Differential Scanning Calorimetry thermogram, which exhibits a significant endo peak about 169° C.

5. A crystalline Form-I of Sumatriptan succinate of claim 1 which has a Differential Scanning Calorimetry thermogram substantially as depicted in FIG. (2).

6. A crystalline Form-I of Sumatriptan succinate of claim 1 having identified characteristic bands about 3373, 3101, 2932, 1708, 1566, 1338, 1299, 1270, 1170, 1081, 884 and 638 cm-¹ in Infra red spectrum.

7. A crystalline Form-I of Sumatriptan succinate of claim 1 having an Infra red spectrum substantially as depicted in FIG. (3).

8. A process for the preparation of crystalline Form-I of Sumatriptan succinate, which comprises;
a) treating highly pure Sumatriptan base in a ketone solvent selected from the group consisting of acetone, methyl isobutyl ketone and methyl ethyl ketone; or an ether solvent selected from the group consisting of tetrahydrofuran, diethyl ether, diisopropyl ether and diisobutyl ether, or an ester solvent selected from the group consisting of methyl acetate, ethyl acetate, propyl acetate and butyl acetate, or an alcoholic solvent selected from the group consisting of methanol, propanol, isopropanol, butanol, isobutanol, and mixtures thereof;
b) adding Succinic acid to the reaction mixture;
c) optionally concentrating the reaction mixture;
d) cooling the reaction mixture to a temperature of 0-35° C.; and
e) filtering the isolated solid accompanied by drying the solid at a temperature of 50-100° C. to afford the crystalline Form-I of Sumatriptan succinate.

9. The process as claimed in claim 8 wherein the ketone solvent of step (a) is acetone.

10. The process as claimed in claim 8 wherein the ether solvent is of step (a) is tetrahydrofuran.

11. The process as claimed in claim 8 wherein the ester solvent of step (a) is ethyl acetate.

12. The process according to claim 8 wherein the highly pure Sumatriptan is at least about 99% pure by HPLC.

13. A crystalline Form-II of Sumatriptan succinate.

14. A crystalline Form-II of Sumatriptan Succinate according to claim 13 having X-ray powder diffraction pattern with peaks comprising about 7.320, 18.751, 19.047, 19.966, 26.089, 29.675 and 31.474 two-theta degrees.

15. A crystalline Form-II of Sumatriptan succinate of claim 13 which has an X-ray powder diffraction pattern substantially as depicted in FIG. (4).

16. A crystalline Form-II of Sumatriptan succinate of claim 13 which has a Differential Scanning Calorimetry thermogram, which exhibits a significant major endo peak about 168° C., and minor endo peaks about 122° C. and 160° C.

17. A crystalline Form-II of Sumatriptan succinate of claim 13 which has a Differential Scanning Calorimetry thermogram substantially as depicted in FIG. (5).

18. A crystalline Form-II of Sumatriptan succinate of claim 13 having infrared characteristic bands at about 3358, 3268, 2931, 1707, 1569, 1336, 1301, 1264, 1143, 1092, 884 and 639 cm⁻¹ in Infra red spectrum.

19. A crystalline Form-II of Sumatriptan succinate of claim 13 having an Infrared spectrum substantially as depicted in FIG. (6).

20. A crystalline Form-II of Sumatriptan Succinate according to claim 13 having X-ray powder diffraction pattern with a peak about 7.320 two-theta degrees and a Differential Scanning Calorimetry thermogram, which exhibits a significant major endo peak about 168° C., and minor endo peaks about 122° C. and 160° C.

21. A process for the preparation of a crystalline Form-II of Sumatriptan succinate, which comprises;
a) refluxing highly pure Sumatriptan in an aliphatic/alicyclic hydrocarbon solvent or a halogenated solvent;
b) adding Succinic acid to the reaction mixture;
c) refluxing the reaction mixture with Succinic acid;
d) cooling the reaction mixture after the step (c); and
e) isolating separated solids to afford crystalline Form-II of Sumatriptan succinate.

22. A process as claimed in claim 21 of step (a), wherein the alicyclic hydrocarbon solvent is cyclohexane.

23. A process as claimed in claim 21 wherein the halogenated solvent of step (a) is dichloromethane.

24. A process according to claim 21 wherein the highly pure Sumatriptan is at least about 99% pure by HPLC.

25. (canceled)

26. (canceled)

27. A process for the preparation of highly pure N-Methyl-3-[2-(dimethylamino) ethyl]-1H-Indole-5 methane sulfonamide (Sumatriptan), which comprises;
a. dissolving crude Sumatriptan in acetone to form a clear solution;
b. treating the obtained clear solution with charcoal;
c. concentrating the clear filtered solution to about filterable volume level;
d. cooling the reaction mixture to a temperature of 0-30° C.; and
e. isolating the obtained solid.

28. The process according to claim 27 wherein the highly pure Sumatriptan is at least about 99% pure by HPLC.

29. A composition comprising a crystalline Form I of Sumatriptan succinate as defined as in claim 1 and one of more pharmaceutically acceptable carrier.

30. A composition comprising a crystalline Form II of Sumatriptan succinate as defined as in claim 13 and one of more pharmaceutically acceptable carrier.

31. (canceled)

32. (canceled)

33. (canceled)

34. (canceled)

35. (canceled)

36. A crystalline form of sumatriptan base having a purity of about 99% or higher by HPLC.

37. A crystalline form of sumatriptan base according to claim 36, wherein said purity is about 99.5% or higher by HPLC.

38. A crystalline form of sumatriptan base according to claim 36, wherein said purity is about 99.7% or higher by HPLC.

39. A crystalline form of sumatriptan base according to claim 36, wherein said crystalline form of sumatriptan base has any unknown purity about 0.1 % or less.

40. A crystalline form of sumatriptan base according to claim 36, wherein said crystalline form has an X-ray powder diffraction pattern substantially the same as FIG. 7.

41. A crystalline form of sumatriptan base according to claim 36, wherein said crystalline form has an infrared spectrum substantially the same as FIG. 8.

42. The crystalline Form-II of Sumatriptan succinate according to claim 14, wherein said peaks further comprise 14.707 and 22.904 two-theta degrees.

43. A composition comprising the crystalline form of sumatriptan base as defined as in claim 36 and one or more pharmaceutically acceptable carrier.

44. A method for treating a migraine comprising administering an effective amount of the compound of claim 1.

45. A method for treating a migraine comprising administering an effective amount of the compound of claim 15.

46. A method for treating a migraine comprising administering an effective amount of the compound of claim 36.

47. A compound of sumatriptan base prepared according to claim 27.
